(19) United States
(12) Patent Application Publication
(10) Pub. No.: US 2013/0183736 A1
(43) Pub. Date: Jul. 18, 2013

(54) Process for Designing, Constructing, and Characterizing Fusion Enzymes for Operation in an Industrial Process

(71) Applicant: Lawrence W. Cosenza, Germantown, NY (US)

(72) Inventor: Lawrence W. Cosenza, Germantown, NY (US)

(73) Assignee: C2 BIOTECHNOLOGIES, LLC, Germantown, NY (US)

(21) Appl. No.: 13/352,699

(22) Filed: Jan. 18, 2012

(51) Int. Cl. C12N 9/00; G06F 17/10

(52) U.S. Cl. 435/183; 703/2

(57) ABSTRACT

A method and a software tool is achieved that measures the distance between a parameter vector that describes an industrial process and vectors that characterize different enzymatic activities. A distance matrix is built and used to construct a hierarchical binary cluster tree of parameter vectors. A novel scoring system is used to rank the hierarchically grouped enzymes. To select the best enzymes for the chimeric fusion enzyme for a particular industrial process, the novel scoring system takes into account biochemical and biophysical variables by creating a belief system. The scoring is generated by summing the products of a biochemical/biophysical variable and its belief parameters/weights for those enzymes found by the distance matrix to have enzymatic activities closest to the enzymatic activities of the industrial process. These scores are then used to select the best enzymes for the particular industrial process.

### RELATED PATENT APPLICATION

This patent application is related to patent application Ser. No. 12/157,686, filed on Jun. 12, 2008, and assigned to the same assignee as the present invention, and incorporated herein in its entirety.

### BACKGROUND OF THE INVENTION

(1) Field of the Invention

The invention relates to a method for designing and constructing fusion enzymes for use in an industrial process, and more particularly, to a method and a tool for optimizing enzymatic activities for use in industry.

(2) Description of the Related Art

A small market study has identified a need for more robust products in the ethanol fuel industry. In particular, the study identified a need for amylase enzymes that are catalytically active at a relatively wide range of temperatures between 70° C. and 100° C.

In general, enzymes work optimally as catalysts within a relatively narrow range of physical parameters such as pH or temperature. Often, but not always, these ranges reflect the normal physiological ranges to be encountered by the organism that produces the enzyme. However, it is difficult in an industrial setting to maintain the enzyme catalyst at its environmental optimum due to the constraints of the industrial process itself, issues of scaling, the particular equipment used, or considerations of quality control.

An example of an enzyme of great industrial utility is a-amylase, used since the 1970s for the “liquefaction” of starch, i.e. the hydrolysis of starch to glucose (Ingle and Erickson, 1978). A typical protocol for the high-temperature, liquid-phase enzymatic hydrolysis of starch involves cooking a starch slurry in the presence of a-amylase at 90-165° C., cooling if necessary and holding the slurry at 90° C. for 1-3 hours, then cooling further to 60° C. with the addition of glucoamylase (Robertson et al., 2006). As manufacturers strive to increase the energy efficiency of the production of fuel ethanol from starch, great interest has been generated in lowering starch-to-glucose processing temperatures into the 54-65° C. range (Robertson et al., 2006). It is doubtful that any single a-amylase purified from a single organism would be equally active at such a wide range of temperatures. Enzymes purified from various sources exhibit temperature optima ranging from 30° C. for the a-amylase of the psychrophilic bacterium Alteromonas haloplanctis, to 50° C. for pig pancreatic a-amylase, to 80° C. for the enzyme of the mesophilic bacterium Bacillus amyloliquefaciens, to 100° C. for the a-amylase of the hyperthermophilic archaeon Pyrococcus woesi (Fitter, 2005). Because of their industrial importance, the structural bases for the thermostability of the various a-amylases active at elevated temperatures have been studied extensively (Zeikus et al., 1998; Fitter, 2005). The various enzymes also exhibit various pH optima, ranging from 5.0-6.5; industrial liquefaction of starch is usually run at pH 6.5 (Synowiecki et al., 2006).

In the example discussed here, it is clear that enzymes from different sources would have to be added to the different steps in the industrial process to try to match the enzyme to the physical conditions of temperature and pH for each step. Therefore, in order to accommodate changes in physical parameters such as temperature or pH in enzyme-catalyzed industrial processes, genes will be constructed encoding chimeric fusion enzymes, proteins with two or more functional active sites from enzymes whose individual parameter optima in aggregate span the desired range. Such a gene will be inserted into an appropriate expression vector along with a targeting sequence that will ensure that the expressed enzyme is secreted from the cell in which it is made and along with proprietary security sequences that will unambiguously identify the construct. Appropriate cells will be transformed with the expression vector and these cells will be induced to express the fusion gene, secreting it into their environment. The chimeric enzyme will then be purified from harvested growth medium. The multiple active sites will be fully functional and will catalyze their reactions independently of each other, conferring upon the fusion enzyme activity over a wide range of temperature, pH, or other environmental parameters. The invention described below is a tool that allows for the selection of enzyme activities with reported operational parameters that coincide with an industrial process.

Several patent publications discuss clustering and scoring systems. These include US 2009/0313192 (Baughman et al), disclosing a K-means clustering system, with a discriminant analysis scoring system for evolutionary facial feature selection, US 2009/0113246 (Sabato et al), disclosing a clustering system with a scoring system which uses a dataset of system logs to rank system log messages based on their relevance to administrators, and US 2008/0097820 (Koran et al), disclosing an unsupervised clustering process and scoring system to identify particular attributes of a set of records that are most associated with “good” records.

### SUMMARY OF THE INVENTION

A primary objective of the invention is to provide a process for designing a fusion enzyme for an industrial process.

Another objective of the invention is to provide a method and a tool for optimizing enzymatic activities for industrial processes.

Another objective is to provide a method and a tool for constructing a database that is composed of data encoding enzyme activity that has been fused with data describing enzyme physical attributes.

Yet another objective is to provide a method and a tool for scoring or ranking enzymes in a database that is composed of data encoding enzyme activity that has been fused with data describing enzyme physical attributes.

The present invention is a method and a software tool that measures the distance between a parameter vector that describes an industrial process and vectors that characterize different enzymatic activities. A distance matrix is built and used to construct a hierarchical binary cluster tree of parameter vectors. A novel scoring system is used to rank the hierarchically grouped enzymes. To select the best enzymes for the chimeric fusion enzyme for a particular industrial process, the novel scoring system takes into account biochemical and biophysical variables, by creating a belief system. The scoring is generated by summing the products of a biochemical/biophysical variable and its belief parameters/weights. These scores are then used to select the best enzymes for the particular industrial process.

### BRIEF DESCRIPTION OF THE DRAWINGS

In the accompanying drawings forming a material part of this description, there is shown:

FIG. 1 illustrates in graphical representation a K-means cluster analysis comprising a scatter plot using optimal pH, temperature, and stability for cellulases.

FIG. 2 graphically illustrates similarity measures of the industrial process parameter enzyme code of tree exocellulases.

FIG. 3 graphically illustrates similarity measures of the industrial process parameter enzyme code of tree glucosidases.

FIG. 4A illustrates a sample list of biochemical and biophysical variables.

FIG. 4B illustrates sample belief system weighting of the variables in FIG. 4A with sample weights.

FIG. 5A illustrates a sample output after applying the scoring system of the invention to the list of enzymes in FIG. 2.

FIG. 5B illustrates a sample output after applying the scoring system of the invention to the list of enzymes in FIG. 3

### DETAILED DESCRIPTION OF THE PREFERRED EMBODIMENTS

Multiple enzymatic activities required for reducing cellulosic material to fermentable sugars can be combined into one protein molecule; hence potentially reducing the costs associated with manufacturing consumables for renewable energy markets and operating costs for clients.

In order to choose the best two enzymes to be combined for a particular industrial process, a method and a tool have been designed. The tool measures the distance between a parameter vector that describes an industrial process and vectors that characterize exocellulase, endocellulase, and glucosidase enzyme activities. Databases store both parameter vectors that describe industrial processes and vectors that characterize enzyme activities for a variety of enzymes of all types. There are public databases that contain biochemical and biophysical data for many types of enzymes. One example is BRENDA (Scheer 2011).

A distance matrix is built and used to construct a hierarchical binary cluster tree of parameter vectors. This tool allows us to select enzyme activities with reported operational parameters that coincide with an industrial process. The novelty is twofold: first, we are engineering an enzyme for a specific process instead of improving on a specific enzyme's catalytic ability, and second, we use multiple variables or parameters for the selection process. The number of parameters we can use is restricted only by the amount and type of data available. The mathematical modeling can handle as many parameters as can be measured.

A result of this work is the demonstration that the enzymatic activities required for reducing cellulose to fermentable sugar, for example, cluster into different groups. This observation suggests that there may be different mechanisms involved in cellulose degradation. This tool also allows us to identify different enzymatic activities from various organisms that operate in similar environments, hence allowing us to postulate a cocktail of enzymatic activities that will work together.

A potential application for this tool is to optimize the enzymatic activities required for a particular industrial process. Furthermore, use of this tool is to optimize the enzymatic activities required for an industrial process. Furthermore, use of this tool in combination with industrial process models may optimize process costs by identifying the least expensive process parameters where an enzymatic cocktail will function.

To select the top candidate enzymes for the fusion process, a novel scoring schema was constructed that combines biochemical and biophysical variables and a “belief system.” The biochemical and biophysical variables are reported and recorded in the databases mentioned earlier and these data describe or characterize a specific enzyme's activity and structure using protein descriptors. The belief system is used to weight the importance of various biochemical and biophysical data for the industrial process and protein engineering. The belief system weights are input by the user. The scoring is generated by summing the products of a biochemical/biophysical variable and belief parameters/weights. The scores are then used to rank the enzymes that would best form the basis of a fusion enzyme to be constructed for use in an industrial process.

This tool also allows us to identify different enzymatic activities from various organisms that operate in similar environments, hence allowing us to postulate a cocktail of enzymatic activities that will work together.

### EXAMPLE

The following example is given to show the important features of the invention and to aid in the understanding thereof. Variations may be made by one skilled in the art without departing from the spirit and scope of the invention.

For example, cluster analysis of cellulosic enzyme activities suggests different environments and possible mechanisms may be used for digesting cellulose. Three databases were constructed that organized operation parameters for three enzymatic activities required for digesting cellulose. The available data allowed us to work in three dimensions. Other dimensions are possible with this type of analysis, but are limited to the available data. A three-dimensional scatter plot of the enzymes' functional variables: pH, temperature, and stability was performed. A K-means clustering algorithm was applied to this information and resulted in displaying the center of several clusters (marked by a +) that occur throughout the scatter plot (See FIG. 1). The cluster centers have different temperature, pH, and time values suggesting that different environments are possible that allow digestion of cellulose. While the number of observations is small and the clusters poorly formed, it is believed that the results suggest that there may be different mechanisms available for digesting cellulose.

The same variables published for a particular process for digesting cellulose were also plotted on the scatter graph to give an indication of the potential enzymes that might be available for testing. This is the industrial process parameter vector. FIG. 1 illustrates the scatter plot and cluster analysis. Point 200 is the desired industrial process temperature, pH, and stability.

The identification of clusters in the scatter plot suggested that building industrial process parameters around center parameters might improve cellulosic biotransformation. This led to the notion that, given an industrial process, we can select enzymes with similar operation parameters. The distance function used during k-means clustering could identify closely-situated enzymes. Closely-situated enzymes could be demonstrated using a hierarchical binary cluster tree. In fact, the distance can be quantified. Henceforth, we can apply this analysis to various enzymatic activities and select the closest related molecules for a specific process. The distance function can use any of a variety of distance functions, including Euclidean distance, standardized Euclidean distance, city block distance, Mihalanobis distance, and Minkowski distance, as well as any other distances.

In this example, an industrial process parameter vector (IPP) was created and used to identify clusters of exocellulase and glucosidase enzymes as potential fusion targets. We have selected an exocellulase and a glucosidase as targets for our fusion process. It will be understood that in other examples, different types of enzymes might be more desirable. An IPP vector was constructed using parameters consistent with simultaneous scarification and fermentation (SSF). In this example, the parameters are temperature, pH, and stability, but a user can choose other parameters such as salinity, pressure, and so on.

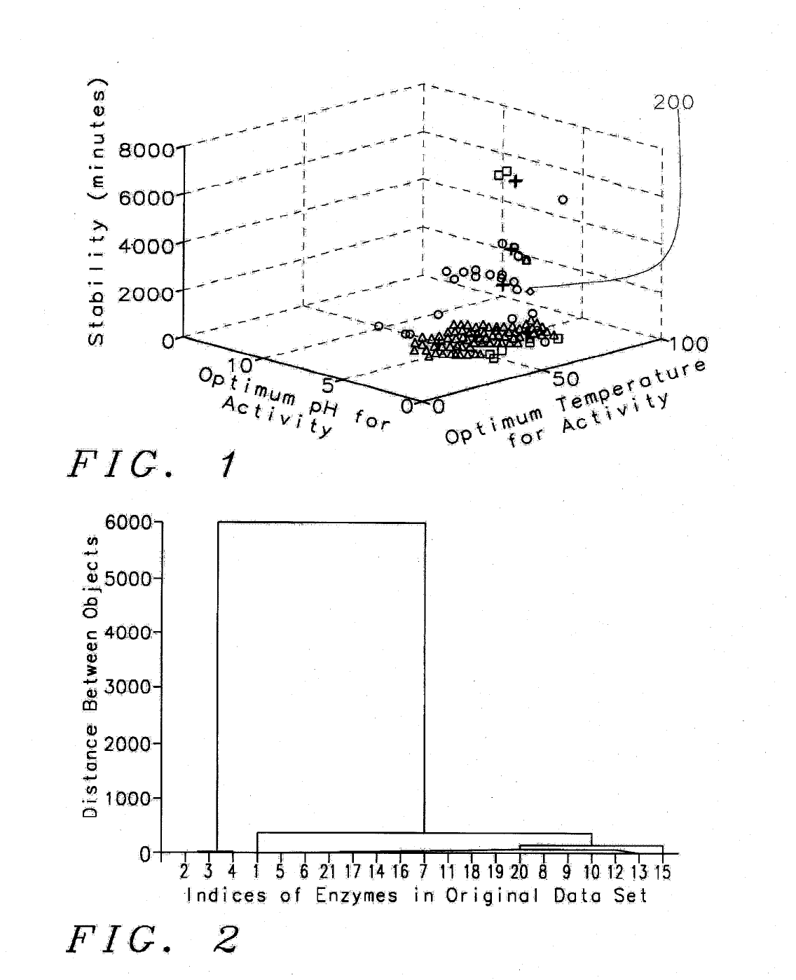

The SSF IPP vector was used to identify two groups of related enzymatic activity. The composition of each group formed a list of exocellulases and glucosidases. FIG. 2 is a tree representation of the distance matrix for cellulases and FIG. 3 is a tree representation for glucosidases. The distance matrix is calculated for each group of enzyme activities. Both distance matrixes use the same IPP. So we are looking at how close the optimal enzyme operating conditions are to the IPP. In FIGS. 2 and 3, index 1 along the horizontal axis represents the IPP vector. The other indices are enzymes to be considered. The figures show the distance between each of the enzymes and the IPP vector, representing the desired industrial parameter values. For example, in FIG. 2, enzymes 2, 3, and 4 are the farthest away from the IPP 1. All the other enzymes in FIG. 2 are equally close to IPP 1.

Table 1 lists the cellulases that correspond to each of the indices in FIG. 2.

TABLE 1
Vector/Cellulase Source and Index

| Vector Name | Index |
|---|---|
| IPP | 1 |
| Clos. thermocellum | 2 |
| Clos. thermocellum | 3 |
| Clos. thermocellum | 4 |
| Geotrichum sp. | 5 |
| Irpex lacteus | 6 |
| Penicillium sp. | 7 |
| T. emersonii | 8 |
| T. emersonii | 9 |
| T. emersoni | 10 |
| Trichoderma viride | 11 |
| a. acidocaldarious | 12 |
| Acidothermus cellulolyticus | 13 |
| Paenibacillus sp. | 14 |
| Thermotoga sp. | 15 |
| Bact. succinogenes | 16 |
| Sclerotium rolfsii | 17 |
| Ruminococcus albus | 18 |
| Bacillus subtilis | 19 |
| Phan. chrysosporium | 20 |
| Aspergillus niger | 21 |

For example, 2, 3, and 4 are the same organism. A single organism can produce many different cellulases, having the same activity, but different amino acid composition. The cellulases may work in different environments or at different pHs, temperatures, and so on. Some cellulases may be membrane attached or secreted into the environment.

Table 2 lists the glucosidases that correspond to each of the indices in FIG. 3.

TABLE 2
Vector Glucosidase Source and Index

| Vector Name | Index |
|---|---|
| IPP | 1 |
| Agrobac. tumefaciens | 2 |
| Agroba. tumefaciens | 3 |
| Aspe aculeatus | 4 |
| Aspe aculeatus | 5 |
| Aspe aculeatus | 6 |
| Aspe aculeatus | 7 |
| Aspe japonicus | 8 |
| Aspergillus niger | 9 |
| Aspergillus niger | 10 |
| Aspergillus niger | 11 |
| Aspergillus niger | 12 |
| Aspergillus oryzae | 13 |
| Aspergillus oryzae | 14 |
| Aspergillus oryzae | 15 |
| Asper. tubingensis | 16 |
| Aspergillus wentii | 17 |
| Aspergillus wentii | 18 |
| Aspergillus wentii | 19 |
| Aureo. pullulans | 20 |
| Avena sativa | 21 |
| Bifidoba. animalis | 22 |
| Bifido. breve | 23 |
| Bombyx mori | 24 |
| Camellia sinensis | 25 |
| Cand. pelliculosa | 26 |
| Candida peltata | 27 |
| Candida sake | 28 |
| Candida wickerhamii | 29 |
| Carica papaya | 30 |
| Cellu. biazotea | 31 |
| Cellu. flavigena | 32 |
| Cellvibrio gilvus | 33 |
| Chae. thermophilum | 34 |
| Chalara paradoxa | 35 |
| Citrus sinensis | 36 |
| Citrus sinensis | 37 |
| Clost. stercorarium | 38 |
| Clost. thermocellum | 39 |
| D. cochinchinensis | 40 |
| Dald. eschscholzii | 41 |
| Debary. vanrijiae | 42 |
| Digitalis lanata | 43 |
| Dioscorea caucasica | 44 |
| Fusarium oxysporum | 45 |
| Glycine max | 46 |
| Glycine max | 47 |
| Hanse. uvarum | 48 |
| Homo sapiens | 49 |
| Homo sapiens | 50 |
| Hordeum vulgare | 51 |
| Humicola grisea | 52 |
| Humicola sp. | 53 |
| Lacto. acidophilus | 54 |
| Microbispora | 55 |
| Mucor miehei | 56 |
| Neot. koshunensis | 57 |
| Olea europaea | 58 |
| Olea europaea | 59 |
| Paecilomyces sp. | 60 |
| Paecil. thermophila | 61 |
| Paenibacillus sp. | 62 |
| Pecto. carotovorum | 63 |
| Pen. aurantiogriseum | 64 |
| Pen. decumbens | 65 |
| Phan. Chrysosporium | 66 |
| Phyto. infestans | 67 |
| Pichia/S. cerevisiae | 68 |
| Pichia etchellsii | 69 |
| Pichia pastoris | 70 |
| Plumeria obtusa | 71 |
| Podo. peltatum | 72 |
| Pseudo. sp. ZD-8 | 73 |
| Pyro furiosus | 74 |
| Rhizobium trifolii | 75 |
| R albus | 76 |
| Sacch. Cerevisiae | 77 |
| Sacch. Fibuligera | 78 |
| Sacch. fibuligera | 79 |
| Sclero sclerotiorum | 80 |
| Sclero sclerotiorum | 81 |
| Sclero sclerotiorum | 82 |
| Sclerotium rolfsii | 83 |
| Scytalidium lignicola | 84 |
| Stachybotrys sp. | 85 |
| Talaro. thermophilus | 86 |
| Thermo aurantiacus | 87 |
| Thermo aurantiacus | 88 |
| Thermo aurantiacus | 89 |
| Thermo aurantiacus | 90 |
| Thermo. Lanuginosus | 91 |
| Therm. thermophilus | 92 |
| T. reesei | 93 |
| T. reesei | 94 |
| Volva. volvacea' | 95 |
| Zea mays' | 96 |

The use of exocellulase and glucosidase in the example is advantageous because the product of exocellulase is a substrate for glucosidase and because the pretreatment of cellulosic feed stocks essentially opens up cellulose for hydrolysis, in effect doing the work of an endocellulase. However, it will be understood that in other industrial processes, other types of enzymes may be more advantageous.

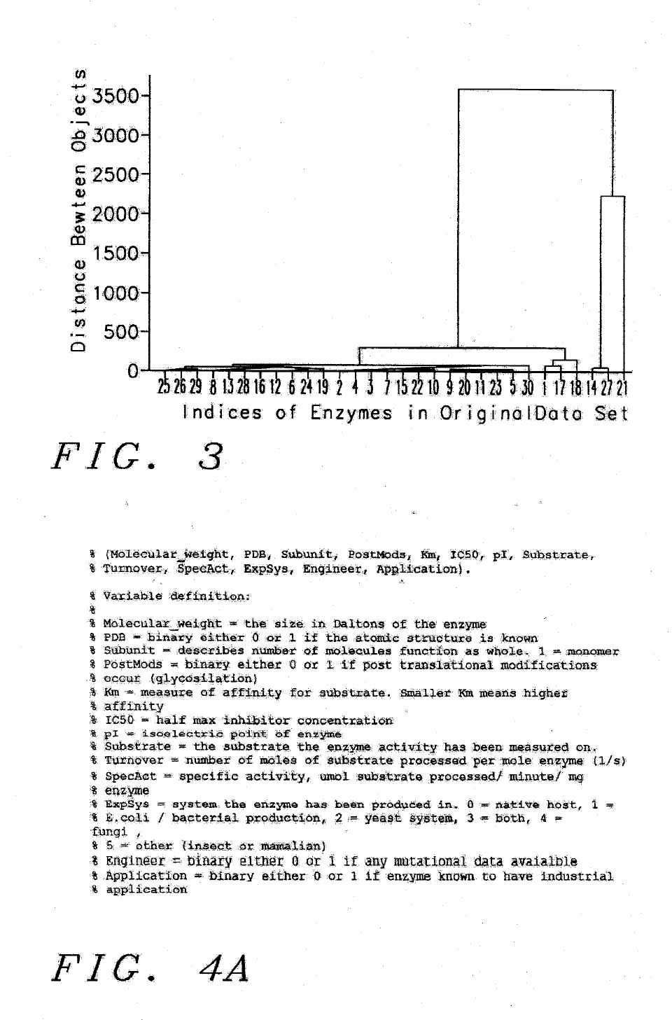

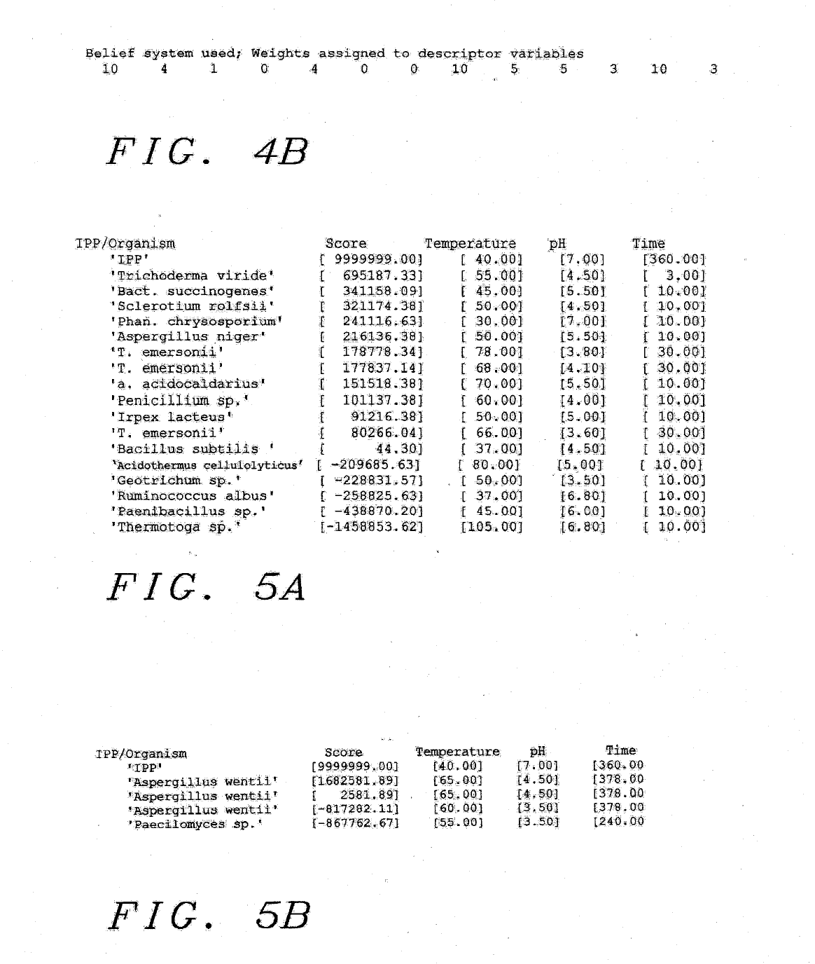

Each group of enzymes was ranked based upon our novel priority scoring function. The scoring function took into account various biochemical, enzymatic, and structural parameters along with other factors considered important for engineering. Each scoring factor was multiplied by a weight; these weights formed the basis for our belief system. FIG. 4A lists thirteen biochemical and biophysical properties of enzymes used in this example. These are the scoring factors.

FIG. 4B shows the belief system vector of thirteen numbers or weights corresponding to the thirteen enzyme descriptors in FIG. 4A. The sum of the weighted protein descriptors formed the score for individual enzymes.

Working in the Matlab (c. 1994-2010 MathWorks, Inc., 3 Apple Hill Drive, Natick, Mass. 01760) programming environment allowed us to easily handle missing data. In effect, enzymes that were better described resulted in better scores. The ability to input a vector that represents a belief system allows the user to selectively emphasize protein descriptors important for selection. For example, a higher affinity measure for the substrate (Km) resulted in a larger score. The belief system can also be used to reduce scoring for unfavorable variables. Experience in protein engineering can also be incorporated into the belief system. For example, the physical size of a protein can be important for stability. The average molecular mass of cellulase was calculated. The actual mass of a cellulase enzyme was subtracted from the calculated average mass. If the cellusase mass was smaller than the average, a positive score was generated and if the specific mass was larger, a negative score was generated. Hence, a smaller cellulase molecule would score better than a larger enzyme. The actual difference is then weighted by the belief system.

FIG. 5A illustrates an example of the result of applying the scoring system of the invention to the list of enzymes in FIG. 2 to be considered; that is, those enzymes that were close to IPP 1. The first row in the output is the industrial process parameter (IPP) vector. Its score is a place holder. The score is calculated for each enzyme by summing the products of belief system weight for each biochemical/biophysical variable used. FIG. 4A defines the variables used in this project and FIG. 4B defines the weights used for this project For example belief system weight #1 (the number 10) corresponds with biophysical data molecular weight. The order in which the variables are defined corresponds to the order of weights in the belief system.

Similarly, FIG. 5B illustrates an example of the result of applying the scoring system of the invention to the list of enzymes in FIG. 3 to be considered; that is, those enzymes that were close to IPP 1. In FIG. 3, enzymes 14, 27 and 21 are the farthest away. Indices 17 and 18 are the closest to index 1. The rest of the indices are equally close to 1. In FIG. 5B, only the top scoring enzymes are shown.

Our novel clustering and scoring system was used to select an exocellulase and a glucosidase sequence for fusion. The top-ranking enzyme sequences in each of FIG. 5A and FIG. 5B were selected for synthesis and the fusion process.

Based on three-dimensional protein crystallization structure data of related enzymes, the sequences were arranged so as to mitigate any interaction or interference with proposed regions responsible for enzymatic activity or substrate building. A linker sequence and security sequence were inserted between the sequences. Native signal and translocation sequences were removed. The resultant fusion enzyme amino acid sequence was then reverse translated into a DNA sequence. The fusion gene sequence was subjected to an endonuclease restriction enzyme digestion analysis to identify restriction sites. Restriction sites not wanted were removed by mutating the DNA sequence so that a different codon was constructed that encoded for the same amino acid. In this fashion, we could alter the primary DNA structure without changing the corresponding amino acid sequence. Unique restriction sides were introduced at appropriate locations to facilitate cloning into our expression vector and for potential future cassette mutagenesis. The expression vector we have chosen to work with is pHT43. The pHT43 expression vector was designed to operate in E. coli and Bacillus and has multiple cloning sites with a signal sequence that directs the expression of target genes to the culture medium.

In summary, the process and tool of the invention can be used to:
1) For a variety of enzymes, record biochemical and biophysical variables that describe or characterize a specific enzyme's activity and structure
2) Use a distance function to determine how close the optimal enzyme operating conditions are to the IPP.
3) Create a belief system used to weight the importance of the various biochemical and biophysical data for the industrial process and protein engineering, based on user-defined weighting.
4) For those enzymes determined to be closest to the IPP (by the distance function), sum the products of the biochemical/biophysical variables and belief parameters/weights to result in a score.
5) Use the scores to select which enzymes would best form the basis of a fusion enzyme to be constructed for use in an industrial process.

### LITERATURE CITED

Deutcher, M. P., 1990, Guide to Protein Purification, Methods in Enzymology, Vol. 182: San Diego, Academic Press.
Fitter, J., 2005, Structural and dynamical features contributing to thermostability in α-amylases: Cellular and Molecular Life Sciences, v. 62, no. 17, p. 1925-1937.
Glick, B. R., and J. J. Pasternak, 1994, Molecular Biotechnology: Principles and Applications of Recombinant DNA, Washington, D.C., ASM Press.
Ingle, M. B., and R. J. Erickson, 1978, Bacterial α-amylases: Advances in Applied Microbiology, v. 24, p. 257-278.
Robertson, G. H., D. W. S. Wong, C. C. Lee, K. Wagschal, M. R. Smith, and W. J. Orts, 2006, Native or raw starch digestion: a key step in energy efficient biorefining of grain: Journal of Agricultural and Food Chemistry, v. 54, no. 2, p. 353-365.
Sambrook, J., E. F. Fritsch, and T. Maniatis, 1989, Molecular Cloning: A Laboratory Manual, 2nd ed., Vol. 1-3: Cold Spring Harbor, N.Y., Cold Spring Harbor Laboratory.
Scopes, R. K., 1994, Protein Purification: Principles and Practice, 3rd ed., New York, Springer-Verlag.
Scheer M., Grote A., Chang A., Schomburg I., Munaretto C., Rother M., Söhngen C., Stelzer M., Thiele J., Schomburg D., 2011, BRENDA, the enzyme information system in 2011. Nucleic Acids Res., 39:670-676.
Synowiecki, J., B. Grzybowska, and A. Zdzieblo, 2006, Sources, properties and suitability of new thermostable enzymes in food processing: Critical Reviews in Food Science and Nutrition, v. 46, no. 3, p. 197-205.
Zeikus, J. G., C. Vieille, and A. Savchenko, 1998, Thermozymes: Biotechnology and structure-function relationships: Extremophiles, v. 2, no. 3, p. 179-183.

Any patents, applications or publications mentioned in the specification are indicative of the levels of those skilled in the art to which the invention pertains. These patents, applications and publications are herein incorporated by reference to the same extent as if each individual publication was specifically and individually indicated to be incorporated by reference.

In view of the teaching presented herein, other modifications and variations of the present invention will readily be apparent to those of skill in the art. The discussion and description are illustrative of some embodiments of the present invention, but are not meant to be limitations on the practice thereof. It is the following claims, including all equivalents, which define the scope of the invention.

What is claimed is:

1. A method to choose the enzymes for a chimeric fusion enzyme for an industrial process comprising the steps of:
plotting data comprising enzyme activities of a plurality of enzymes for a certain number of activities;
creating an industrial process parameter vector (IPP) identifying a target value of said certain number of activities;
performing a K-means clustering algorithm on a scatter plot of said data;
using a distance function to identify enzymes closely-situated to said IPP target value;
ranking each group of closely-situated enzymes based upon a novel priority scoring function; and
choosing the best available enzyme in each group, based on said novel priority scoring function, for said chimeric fusion enzyme for said industrial process.

2. The method of claim 1 wherein said K-means clustering algorithm displays the center of several clusters that occur throughout the scatter plot.

3. The method of claim 1 wherein said distance function can use any of a variety of functions, including but not limited to Euclidean distance, standardized Euclidean distance, city block distance, Mihalanobis distance, and Minkowski distance.

4. The method of claim 1 wherein said closely-situated enzymes can be demonstrated using a hierarchical binary clustering tree.

5. The method of claim 1 wherein said groups of enzymes can be endocullulases, exocellulases, or glucosidases.

6. The method of claim 1 wherein said industrial process parameter vector is constructed using parameters consistent with simultaneous scarification and fermentation (S SF).

7. The method of claim 1 wherein said novel scoring system takes into account various biochemical, biophysical, enzymatic, and structural parameters which describe and characterize a specific enzyme's activity and structure using protein descriptors.

8. The method of claim 1 wherein said novel scoring system relies on a belief system, consisting of user-generated weights.

9. The method of claim 2 wherein said clusters have different temperature, pH and time values.

10. The method of claim 4 wherein said hierarchical binary clustering tree allows for the selection of enzyme activities with reported operational parameters that coincide with said industrial process.

11. The method of claim 7 wherein said novel scoring system scores a said enzyme by multiplying said protein descriptors by their corresponding weights, and summing the weighted scores.

12. The method of claim 8 wherein said user-generated weights are chosen so as to selectively emphasize protein descriptors that are important for a specific said industrial process.

13. The method of claim 10 wherein a number of said operational parameters is only limited by the number and type of data available.

14. The method of claim 11 wherein said weighted scores are used to select which enzymes form the basis for said chimeric fusion enzyme.

15. A computer program for choosing the enzymes for a chimeric fusion enzyme for an industrial process, said computer program residing on a non-transitory computer readable medium, comprising the steps of:
performing a K-means clustering algorithm on a scatter plot of data;
using a distance function to identify closely-situated enzymes;
creating an industrial process parameter vector (IPP) used to identify clusters of enzymes as potential fusion targets;
ranking each group of closely-situated enzymes based upon a novel priority scoring function; and
choosing the best available enzyme in each group, based on said novel priority scoring function, for use in said chimeric fusion enzyme for said industrial process.

16. The computer program of claim 15 wherein said K-means clustering algorithm displays the center of several clusters that occur throughout the scatter plot.

17. The computer program of claim 15 wherein said distance function uses any of a variety of functions, including but not limited to Euclidean distance, standardized Euclidean distance, city block distance, Mihalanobis distance, and Minkowski distance.

18. The computer program of claim 15 wherein said closely-situated enzymes can be demonstrated using a hierarchical binary clustering tree.

19. The computer program of claim 15 wherein said novel scoring system takes into account various biochemical, biophysical, enzymatic, and structural parameters which describe and characterize a specific enzyme's activity and structure using protein descriptors and stored in a first database.

20. The computer program of claim 19 wherein said novel scoring system relies on a belief system, consisting of user-generated weights, and stored in a second database.

21. The computer program of claim 19 wherein said novel scoring system scores a said enzyme by multiplying said protein descriptors by their corresponding weights, and summing the weighted scores.

22. The computer program of claim 20 wherein said user-generated weights are chosen so as to selectively emphasize protein descriptors that are important for a specific said industrial process.

23. A method of manufacturing a fusion enzyme for an industrial process, comprising the steps of:
selecting an industrial process parameter;
selecting two or more enzymes, produced by two or more respective organisms, having biological activity based on said industrial process parameter, said selecting comprising the steps of:
performing a K-means clustering algorithm on a scatter plot of data;
creating an industrial process parameter vector (IPP) used to identify clusters of enzymes as potential fusion targets;
using a distance function to identify closely-situated enzymes;
ranking each group of closely-situated enzymes based upon a novel priority scoring function; and
choosing the best available enzyme in each group, based on said novel priority scoring function, for said industrial process; and
forming said fusion enzyme by genetically linking said two or more enzymes together.

24. The method of claim 23 wherein said closely-situated enzymes can be demonstrated using a hierarchical binary clustering tree.

25. The method of claim 23 wherein said novel scoring system takes into account various biochemical, biophysical, enzymatic, and structural parameters which describe and characterize a specific enzyme's activity and structure using protein descriptors.

26. The method of claim 23 wherein said novel scoring system relies on a belief system, consisting of user-generated weights.

27. The method of claim 25 wherein said novel scoring system scores a said enzyme by multiplying said protein descriptors by their corresponding weights, and summing the weighted scores.

28. The method of claim 26 wherein said user-generated weights are chosen so as to selectively emphasize protein descriptors that are important for a specific said industrial process.

29. A method to choose the enzymes for a chimeric fusion enzyme for an industrial process comprising the steps of:
for a variety of enzymes, record biochemical and biophysical variables that describe or characterize a specific enzyme's activity and structure;
using a distance function, determine how close the optimal enzyme operating conditions are to conditions of said industrial process;
create a belief system used to weight the importance each of said various biochemical and biophysical variables for said industrial process, based on user-defined weighting;
for those enzymes determined by said distance function to be closest to said conditions of said industrial process, generating scores by summing products of said biochemical and biophysical variables and said belief system weights; and
using said scores to select which enzymes would best form the basis of a fusion enzyme to be constructed for use in said industrial process.
